(19) United States
(12) Patent Application Publication
(10) Pub. No.: US 2008/0299474 A1
(43) Pub. Date: Dec. 4, 2008

(54) HIGH QUALITY SUBSTITUTED ARYL DIAMINE AND A PHOTORECEPTOR

(75) Inventors: Gregory MCGUIRE, Mississauga (CA); Jennifer A. COGGAN, Cambridge (CA); Hany AZIZ, Oakville (CA); Johann JUNGINGER, Toronto (CA); Nan-Xing HU, Oakville (CA); Ah-Mee Hor, Mississauga (CA)

Correspondence Address: OLIFF & BERRIDGE, PLC., P.O. BOX 320850, ALEXANDRIA, VA, 22320-4850, US

(73) Assignee: XEROX CORPORATION, Stamford, CT (US)

(21) Appl. No.: 11/756,109

(22) Filed: May 31, 2007

(51) Int. Cl. G03G 15/02; C07C 211/54

(52) U.S. Cl. 430/58.8; 564/306

(57) ABSTRACT

A high quality hole transport material of a substituted biphenyl diamine, such as N,N,N′N′-tetra(4-methylphenyl)-(1,1′-biphenyl)-4,4′-diamine, where each X is independently selected from —H, alkyl(-CₙH₂ₙ₊₁), where n is from 1 to about 10 such as from 1 to about 5 or from 1 to about 6, aralkyl and aryl groups, the aralkyl and aryl groups having, for example, from about 5 to about 30, such as about 6 to about 20 carbon atoms, where the purity of the hole transport material is from about 80 percent to about 100 percent, and when incorporated into a photoreceptor, the photoreceptor discharges from about 85% to about 100% of its surface potential in from 0 to about 40 milliseconds upon being subjected to xerographic charging and exposure to radiant energy of from about 1 ergs/cm² to about, 5 ergs/cm². Also disclosed is a photoreceptor containing the same.

### CROSS-REFERENCE TO RELATED APPLICATIONS

There is illustrated in U.S. patent application Ser. No. 128,327, to Aziz, et al., filed concurrently herewith, the entire disclosure of which is totally incorporated herein by reference, a photoreceptor comprising a substrate, a charge generating layer, a charge transport layer comprising N,N,N′N′-tetra(4-methylphenyl)-(1,1′-biphenyl)-4,4′-diamine having a purity of from about 95 percent to about 100 percent; and a protective overcoating layer optionally comprising a hole transport material other than N,N,N′N′-tetra(4-methylphenyl)-(1,1′-biphenyl)-4,4′-diamine; wherein the photoreceptor will discharge from about 85% to about 100% of its surface potential in from 0 to about 40 milliseconds upon being subjected to xerographic charging and exposure to radiant energy of from about 1 erg/cm² to about 5 ergs/cm².

### REFERENCES

Layered photoresponsive imaging members have been described in numerous U.S. patents, such as U.S. Pat. No. 4,265,990, wherein there is illustrated an imaging member comprised of a photogenerating layer, and an aryl amine hole transport layer. Examples of photogenerating layer components include trigonal selenium, metal phthalocyanines, vanadyl phthalocyanines, and metal free phthalocyanines. Additionally, there is described in U.S. Pat. No. 3,121,006, the disclosure of which is totally incorporated herein by reference, a composite xerographic photoconductive member comprised of finely divided particles of a photoconductive inorganic compound and an amine hole transport dispersed in an electrically insulating organic resin binder.

There is illustrated in U.S. Pat. No. 6,913,863, a photoconductive imaging member comprised of a hole blocking layer, a photogenerating layer, and a charge transport layer, and wherein the hole blocking layer is comprised of a metal oxide; and a mixture of a phenolic compound and a phenolic resin wherein the phenolic compound contains at least two phenolic groups.

In U.S. Pat. No. 4,555,463, is illustrated a layered imaging member with a chloroindium phthalocyanine photogenerating layer.

In U.S. Pat. No. 4,587,189, there is illustrated a layered imaging member with, for example, a perylene, pigment photogenerating component. Both of the aforementioned patents disclose an aryl amine component, such as N,N′-diphenyl-N,N′-bis(3-methylphenyl)-1,1′-biphenyl-4,4′-diamine dispersed in a polycarbonate binder as a hole transport layer. The above components, such as the photogenerating compounds and the aryl amine hole transport molecules, can be selected for the imaging members of the present disclosure in embodiments thereof.

Illustrated in U.S. Pat. Nos. 6,255,027; 6,177,219, and 6,156,468, are, for example, photoreceptors containing a hole blocking layer of a plurality of light scattering particles dispersed in a binder, reference for example, Example I of U.S. Pat. No. 6,156,468, the disclosure of which is totally incorporated herein by reference, wherein there is illustrated a hole blocking layer of titanium dioxide dispersed in a specific linear phenolic binder of VARCUM™, available from OxyChem Company.

Illustrated in U.S. Pat. No. 5,521,306, is a process for the preparation of Type V hydroxygallium phthalocyanine photogenerating pigments comprising the in situ formation of an alkoxy-bridged gallium phthalocyanine dimer, hydrolyzing the dimer to hydroxygallium phthalocyanine, and subsequently converting the hydroxygallium phthalocyanine product to Type V hydroxygallium phthalocyanine.

Illustrated in U.S. Pat. No. 5,482,811, is a process for the preparation of hydroxygallium phthalocyanine photogenerating pigments which comprises hydrolyzing a gallium phthalocyanine precursor pigment by dissolving the hydroxygallium phthalocyanine in a strong acid and then reprecipitating the resulting dissolved pigment in basic aqueous media; removing any ionic species formed by washing with water, concentrating the resulting aqueous slurry comprised of water and hydroxygallium phthalocyanine to a wet cake; removing water from said slurry by azeotropic distillation with an organic solvent, and subjecting said resulting pigment slurry to mixing with the addition of a second solvent to cause the formation of said hydroxygallium phthalocyanine polymorphs.

There is disclosed in U.S. Pat. No. 4,306,008, imaging or photosensitive members with at least two electrically operative layers of a photoconductive layer and a charge transport layer containing a polycarbonate resin and from about 25 to about 75 percent by weight of a substituted aryl diamine, of the formula recited in the abstract and column 6.

The disclosures of each of the foregoing patents are hereby incorporated by reference herein in their entireties. The appropriate components and process aspects of the each of the foregoing patents may also be selected for the present compositions and processes in embodiments thereof.

### BACKGROUND

The high quality hole transport material and photoconductors comprising same illustrated herein in embodiments, possess low post erase voltages; excellent electrical cycling stability; LCM (lateral charge migration) resistance; excellent wear resistance; extended lifetimes; provide for elimination or minimization of imaging member scratches on the surface layer or layers of the imaging member, and which scratches can result in undesirable print failures where, for example, the scratches are visible on the final prints generated. Additionally, in embodiments the imaging members disclosed herein possess an excellent rate of discharge, and in a number of instances low Vᵣ (residual potential), and allow the substantial prevention of Vᵣ cycle up when appropriate; high sensitivity; low acceptable image ghosting characteristics; and desirable toner cleanability.

### SUMMARY

Disclosed is a high quality hole transport material which is a substituted biphenyl diamine of the following formula

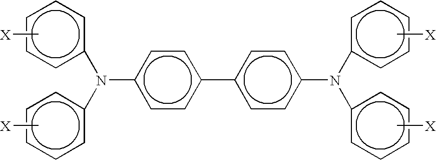

such as N,N,N′N′-tetra(4-methylphenyl)-(1,1′-biphenyl)-4,4′-diamine, where each X is independently selected from the group consisting of —H, alkyl(—CₙH₂ₙ₊₁), where n is from 1 to about 10 such as from 1 to about 5 or from 1 to about 6, aralkyl and aryl groups, the aralkyl and aryl groups having, for example, from about 5 to about 30, such as about 6 to about 20, carbon atoms; and where the purity of the hole transport material is from about 95 percent to about 100 percent, and when incorporated into a photoreceptor, the photoreceptor will discharge from about 85% to about 100% of its surface potential in from 0 to about 40 milliseconds upon being subjected to xerographic charging and exposure to radiant energy of about 1 erg/cm² to about 5 ergs/cm² Also disclosed is a photoreceptor containing the same.

The photoreceptors disclosed herein have many advantages and improvements, such as extended lifetimes of service of, for example, in excess of about 3,500,000 imaging cycles; excellent electronic characteristics; excellent rate of discharge; stable electrical properties; low image ghosting; resistance to charge transport layer cracking upon exposure to the vapor of certain solvents; excellent surface characteristics; improved wear resistance; compatibility with a number of toner compositions; the avoidance of or minimal imaging member scratching characteristics; consistent Vᵣ (residual potential) that is substantially flat or no change over a number of imaging cycles as illustrated by the generation of known PIDC (Photo-Induced Discharge Curve), and the like.

Moreover, disclosed are layered belt photoresponsive or photoconductive imaging members with mechanically robust and solvent resistant charge transport layers.

Additionally disclosed are flexible imaging members with optional hole blocking layers comprised of metal oxides, phenolic resins, and optional phenolic compounds, and which phenolic compounds contain from about two to about twenty, and more specifically, two to ten phenol groups or phenolic resins with, for example, a weight average molecular weight ranging from about 500 to about 3,000, permitting, for example, a hole blocking layer with excellent efficient electron transport which usually results in a desirable photoconductor low residual potential V_{low}.

### EMBODIMENTS

Aspects of the present disclosure relate to improved high quality hole transport components, such as high quality substituted biphenyl diamines. In embodiments, the substituted biphenyl diamines can be represented by the following general formula:

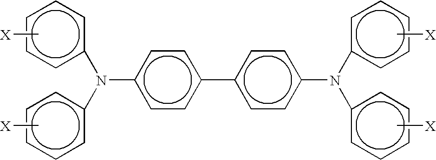

such as N,N,N′N′-tetra(4-methylphenyl)-(1,1′-biphenyl)-4,4′-diamine, wherein each X is independently selected from the group consisting of —H, —OH, alkyl(-CₙH₂ₙ₊₁) where n is from 1 to about 10 such as from 1 to about 5 or from 1 to about 6, aralkyl, and aryl groups, the aralkyl and aryl groups having, for example, from about 5 to about 30, such as about 6 to about 20, carbon atoms. Suitable examples of aralkyl groups include, for example, —CₙH₂ₙ-phenyl groups where n is from 1 to about 5 or from 1 to about 10. Suitable examples of aryl groups include, for example, phenyl, naphthyl, biphenyl, and the like. Alkyl contains for example, from 1 to about 25 carbon atoms, from 1 to about 16 carbon atoms, from 1 to about 10 carbon atoms, or from 1 to about 6 carbon atoms, such as methyl, ethyl, propyl, butyl, pentyl, heptyl, hexyl, dodecyl, and the like.

More specifically, the present disclosure in embodiments is directed to a substituted biphenyl diamine of high quality, such as N,N,N′N′-tetra(4-methylphenyl)-(1,1′-biphenyl)-4,4′-diamine, such that, when incorporated into a photoreceptor, the photoreceptor will exhibit an improved rate of discharge of its surface potential as well as improved cycling stability. As used herein, the term “cycling stability” refers to lack of change in electrical characteristics during electrophotographic cycling. Improving discharge rate is advantageous because high speed printing applications require a shorter expose to development time within which the photoreceptor must discharge its surface potential. Therefore, photoreceptors exhibiting an improved discharge rate are important in high speed printing applications and the like, and may reduce the overall costs associated with large-scale or commercial printing operations. In embodiments, the photoreceptor may discharge from about 85% to about 100% of its surface potential in from about 0 to about 40 milliseconds upon being subjected to xerographic charging and exposure to radiant energy of from about 1 erg/cm² to about 5 ergs/cm², such as from about 85% to about 100% of its surface potential in from about 0 to about 40 milliseconds of being subjected to xerographic charging and exposure to radiant energy of about 1 erg/cm² to about 4 ergs/cm². As used herein, “high quality” referring to the substituted biphenyl diamine thus refers to a substituted biphenyl diamine that, when incorporated into a photoreceptor, the photoreceptor will discharge from about 90% to about 100% of its surface potential in from 0 to about 40 milliseconds upon being subjected to xerographic charging and exposure to radiant energy of about 1 ergs/cm² to about 3 ergs/cm². In embodiments, a photoreceptor comprising the high quality substituted biphenyl diamine may have a post erase voltage of from about 0 to about 10 volts, from an initial charging voltage of from about 400 to about 1000 volts, when erase energy is about 200 ergs/cm². The substituted biphenyl diamine may also exhibit stable xerographic cycling over 10,000 cycles.

In addition to a high quality substituted biphenyl diamine, the present disclosure in embodiments is directed to a substituted biphenyl diamine of high purity, such as for example, a purity of from about 95 percent to about 100 percent, such as from about 98 percent to about 100 percent, as determined for example, by HPLC, NMR, GC, LC/MS, GC/MS or by melting temperature data, and such that, when incorporated into a photoreceptor; the substituted biphenyl diamine hole transport component is in embodiments present in at least a charge transport layer of a photoreceptor in an amount of from about 1 to about 75 weight percent (number values throughout are intended to include all numbers there between, thus about 1 to 75 includes at least all number values of 1, 2, 3, 4, 5, 6, 7 up to 75), about 25 to about 75 weight percent; about 30 to about 65 weight percent, from about 40 to about 60 weight percent, from about 45 to about 55 weight percent; an optional adhesive layer, an optional hole blocking or undercoat layer, and an optional overcoating layer.

Although not limited to any specific theory, it is believed that the high quality of the substituted biphenyl diamine, and the properties provided thereby, may not be directly linked to its chemical purity alone, but instead may be linked to the chemical purity, type and amount of residual impurities, and the like.

Examples of specific substituted biphenyl diamine having the above formulae include N,N′-diphenyl-N,N′-bis(alkylphenyl)-1,1-biphenyl-4,4′-diamine wherein alkyl is selected from the group consisting of methyl, ethyl, propyl, butyl, hexyl, and the like; N,N′-diphenyl-N,N′-bis(halophenyl)-1,1′-biphenyl-4,4′-diamine wherein the halo substituent is a chloro substituent; N,N′-bis(4-butylphenyl)-N,N′-di-p-tolyl-[p-terphenyl]-4,4″-diamine, N,N′-bis(4-butylphenyl)-N,N′-di-m-tolyl-[p-terphenyl]-4,4″-diamine, N,N′-bis(4-butylphenyl)-N,N′-di-o-tolyl-[p-terphenyl]-4,4″-diamine, N,N′-bis(4-butylphenyl)-N,N′-bis-(4-isopropylphenyl)-[p-terphenyl]-4,4″-diamine, N,N′-bis(4-butylphenyl)-N,N′-bis-(2-ethyl-6-methylphenyl)-[p-terphenyl]-4,4″-diamine, N,N′-bis(4-butylphenyl)-N,N′-bis-(2,5-dimethylphenyl)-[p-terphenyl]-4,4′-diamine, N,N′-diphenyl-N,N′-bis(3-chlorophenyl)-[p-terphenyl]-4,4″-diamine, and the like. Other known charge transport layer molecules can be selected, reference for example, U.S. Pat. Nos. 4,921,773 and 4,464,450, the disclosures of which are totally incorporated herein by reference.

Alkyl and alkoxy contain, for example, from 1 to about 25 carbon atoms, and more specifically, from 1 to about 12 carbon atoms, such as methyl, ethyl, propyl, butyl, pentyl, and the corresponding alkoxides. Aryl can contain from 6 to about 36 carbon atoms, such as phenyl, and the like. Halogen includes chloride, bromide, iodide and fluoride. Substituted alkyls, alkoxys, and aryls can also be selected in embodiments.

Aspects of the present disclosure further relate to a photoconductive imaging member comprised of a supporting substrate, a photogenerating layer, a charge transport layer, and an overcoating polymer layer; a photoconductive member with a photogenerating layer of a thickness of from about 0.1 to about 10 microns, and at least one transport layer each of a thickness of from about 5 to about 100 microns; an imaging apparatus containing a charging component, a development component, a transfer component, and a fixing component, and wherein the apparatus contains a photoconductive imaging member comprised of a supporting substrate, and thereover a layer comprised of a photogenerating pigment and a charge transport layer or layers, and thereover an overcoating charge transport layer, and where the transport layer is of a thickness of from about 40 to about 75 microns; a member or photoconductor wherein the photogenerating layer contains a photogenerating pigment present in an amount of from about 5 to about 95 weight percent; a member wherein the thickness of the photogenerating layer is from about 0.1 to about 4 microns; a member wherein the photogenerating layer contains a polymer binder and wherein the binder is present in an amount of from about 50 to about 90 percent by weight, and wherein the total of all layer components is about 100 percent; a member wherein the photogenerating component is a hydroxygallium phthalocyanine that absorbs light of a wavelength of from about 370 to about 950 nanometers; an imaging member wherein the supporting substrate is comprised of a conductive substrate comprised of a metal; an imaging member wherein the conductive substrate is aluminum, aluminized polyethylene terephthalate or titanized polyethylene terephthalate; an imaging member wherein the photogenerating resinous binder is selected from the group consisting of polyesters, polyvinyl butyrals, polycarbonates, polystyrene-b-polyvinyl pyridine, and polyvinyl formals; an imaging member wherein the photogenerating pigment is a metal free phthalocyanine; an imaging member wherein the charge transport layer comprises the substituted biphenyl diamine of the formulae illustrated herein; an imaging member wherein the resinous binder is selected from the group consisting of polycarbonates and polystyrene; an imaging member wherein the photogenerating pigment present in the photogenerating layer is comprised of chlorogallium phthalocyanine, or Type V hydroxygallium phthalocyanine prepared by hydrolyzing a gallium phthalocyanine precursor by dissolving the hydroxygallium phthalocyanine in a strong acid and then reprecipitating the resulting dissolved precursor in a basic aqueous media; removing any ionic species formed by washing with water; concentrating the resulting aqueous slurry comprised of water and hydroxygallium phthalocyanine to a wet cake; removing water from the wet cake by drying; and subjecting the resulting dry pigment to mixing with the addition of a second solvent to cause the formation of the hydroxygallium phthalocyanine; an imaging member wherein the Type V hydroxygallium phthalocyanine has major peaks, as measured with an X-ray diffractometer, at Bragg angles (2 theta+/−0.2°) 7.4, 9.8, 12.4, 16.2, 17.6, 18.4, 21.9, 23.9, 25.0, 28.1 degrees, and the highest peak at 7.4 degrees; a photoconductor comprised of a substituted biphenyl diamine hole transport molecule or molecules and at least one of, for example N,N′-diphenyl-N,N-bis(3-methylphenyl)-1,1′-biphenyl-4,4′-diamine, N,N′-bis(4-butylphenyl)-N,N′-di-p-tolyl-[p-terphenyl]-4,4″-diamine, N,N′-bis(4-butylphenyl)-N,N′-di-m-tolyl-[p-terphenyl]-4,4″-diamine, N,N′-bis(4-butylphenyl)-N,N′-di-o-tolyl-[p-terphenyl]-4,4″-diamine, N,N′-bis(4-butylphenyl)-N,N′-bis-(4-isopropylphenyl)-[p-terphenyl]-4,4″-diamine, N,N′-bis(4-butylphenyl)-N,N′-bis-(2-ethyl-6-methylphenyl)-[p-terphenyl]-4,4″-diamine, N,N′-bis(4-butylphenyl)-N,N′-bis-(2,5-dimethylphenyl)-[p-terphenyl]-4,4″-diamine, N,N′-diphenyl-N,N′-bis(3-chlorophenyl)-[p-terphenyl]-4,4″-diamine molecules, for example in an amount of from about 5 weight percent to about 50 weight percent and wherein the hole transport resinous binder is selected from the group consisting of polycarbonates and polystyrene; a photoconductive imaging member with a blocking layer contained as a coating on a substrate, and an adhesive layer coated on the blocking layer; photoconductive imaging members comprised of a supporting substrate, a photogenerating layer, a hole transport layer and a top overcoating layer in contact with the hole transport layer and in embodiments wherein a plurality of charge transport layers are selected, such as for example, from two to about ten and more specifically two, may be selected; and a photoconductive imaging member comprised of an optional supporting substrate, a photogenerating layer, and a first, second, and third charge transport layer.

In embodiments, at least one charge transport layer is comprised of at least one hole transport component of the above-mentioned formulas/structures. The concentration of the high quality substituted biphenyl diamine hole transport component may be low to, for example, achieve increased mechanical strength and LCM resistance in the photoconductor. In embodiments the concentration of the substituted biphenyl diamine component in the charge transport layer may be from about 10 weight percent to about 65 weight percent and more specifically from about 35 to about 60 weight percent, or from about 45 to about 55 weight percent. The substituted biphenyl diamine component may have a purity of from about 98 percent to about 100 percent, such as from about 99 percent to about 100 percent, and from about 35 weight percent to about 70 weight percent of MAKROLON 5705®, a known polycarbonate resin having a molecular weight average of from about 50,000 to about 100,000, commercially available from Farbenfabriken Bayer A.G.

The charge transport layer, such layer being generally of a thickness of from about 5 microns to about 90 microns, and more specifically, of a thickness of from about 10 microns to about 40 microns, may include a number of hole transport compounds, such as substituted aryl diamines and known hole transport molecules, as illustrated herein, and additional components, including additives, such as antioxidants, a number of polymer binders and the like. In embodiments, additives may include at least one additional binder polymer, such as from 1 to about 5 polymers in a percent weight range of about 10 to about 75 in the charge transport layer; at least one additional hole transport molecule, such as from 1 to about 7, 1 to about 4, or from 1 to about 2 in a percent weight range of about 10 to about 75 in the charge transport layer; antioxidants; like IRGONAX (available from Ciba Specialty Chemical), in a percent weight range of about 0 to about 20, from about 1 to about 10, or from about 3 to about 8 weight percent.

The charge transport layer or layers, and more specifically, a first charge transport layer in contact with the photogenerating layer, and thereover a top or second charge transport overcoating layer may comprise hole transporting small molecules dissolved or molecularly dispersed in a film forming electrically inert polymer such as a polycarbonate. In embodiments, “dissolved” refers, for example, to forming a solution in which the small molecule is dissolved in the polymer to form a homogeneous phase; and “molecularly dispersed in embodiments” refers, for example, to hole transporting molecules dispersed in the polymer, the small molecules being dispersed in the polymer on a molecular scale. Various hole transporting or electrically active small molecules may be selected for the charge transport layer or layers. In embodiments, hole transport refers, for example, to hole transporting molecules as a monomer that allows the free charge generated in the photogenerating layer to be transported across the transport layer.

Examples of added hole transporting molecules, especially for the first and second charge transport layers, include, for example, pyrazolines such as 1-phenyl-3-(4′-diethylamino styryl)-5-(4″-diethylamino phenyl)pyrazoline; aryl amines such as N,N′-diphenyl-N,N′-bis(3-methylphenyl)-(1,1′-biphenyl)-4,4′-diamine, N,N′-bis(4-butylphenyl)-N,N′-di-p-tolyl-[p-terphenyl]-4,4″-diamine, N,N′-bis(4-butylphenyl)-N,N′-di-m-tolyl-[p-terphenyl]-4,4″-diamine, N,N′-bis(4-butylphenyl)-N,N′-di-o-tolyl-[p-terphenyl]-4,4″-diamine, N,N′-bis(4-butylphenyl)-N,N′-bis-(4-isopropylphenyl)-[p-terphenyl]-4,4″-diamine, N,N′-bis(4-butylphenyl)-N,N′-bis-(2-ethyl-6-methylphenyl)-[p-terphenyl]-4,4″-diamine, N,N′-bis(4-butylphenyl)-N,N′-bis-(2,5-dimethylphenyl)-[p-terphenyl]-4,4″-diamine, N,N′-diphenyl-N,N′-bis(3-chlorophenyl)-[p-terphenyl]-4,4″-diamine; hydrazones such as N-phenyl-N-methyl-3-(9-ethyl)carbazyl hydrazone and 4-diethyl amino benzaldehyde-1,2-diphenyl hydrazone; and oxadiazoles such as 2,5-bis(4-N,N′-diethylaminophenyl)-1,2,4-oxadiazole, stilbenes, and the like. However, in embodiments to minimize or avoid cycle-up in equipment, such as printers, with high throughput, the charge transport layer should be substantially free (less than about two percent) of di or triamino-triphenyl methane. A small molecule charge transporting compound that permits injection of holes into the photogenerating layer with high efficiency and transports them across the charge transport layer with short transit times includes N,N′-diphenyl-N,N′-bis(3-methylphenyl)-(1,1′-biphenyl)-4,4′-diamine, N,N′-bis(4-butylphenyl)-N,N′-di-p-tolyl-[p-terphenyl]-4,4″-diamine, N,N′-bis(4-butylphenyl)-N,N′-di-m-tolyl-[p-terphenyl]-4,4″-diamine, N,N′-bis(4-butylphenyl)-N,N′-di-o-tolyl-[p-terphenyl]-4,4″-diamine, N,N′-bis(4-butylphenyl)-N,N′-bis-(4-isopropylphenyl)-[p-terphenyl]-4,4″-diamine, N,N′-bis(4-butylphenyl)-N,N′-bis-(2-ethyl-6-methylphenyl)-[p-terphenyl]-4,4″-diamine, N,N′-bis(4-butylphenyl)-N,N′-bis-(2,5-dimethylphenyl)-[p-terphenyl]-4,4″-diamine, and N,N′-diphenyl-N,N′-bis(3-chlorophenyl)-[p-terphenyl]-4,4″-diamine, or mixtures thereof. If desired, the hole transport material in the charge transport layer may comprise a polymeric hole transport material or a combination of a small molecule hole transport material and a polymeric hole transport material.

Specific examples of a hole transport molecule encompassed by the above formulae may further include a tetra[p-tolyl]biphenyldiamine also referred to as N,N,N′N′-tetra(4-methylphenyl)-(1,1′-biphenyl)-4,4′-diamine; N,N,N′N′-tetra(4-ethylphenyl)-(1,1′-biphenyl)-4,4′-diamine; N,N,N′N′-tetra(4-propylphenyl)-(1,1′-biphenyl)-4,4′-diamine; N,N,N′N′-tetra(4-butylphenyl)-(1,1′-biphenyl)-4,4′-diamine and the like.

Examples of the binder materials selected for the charge transport layer include components, such as those described in U.S. Pat. No. 3,121,006, the entire disclosure of which is totally incorporated herein by reference. Specific examples of polymer binder materials include polycarbonates, polyarylates, acrylate polymers, vinyl polymers, cellulose polymers, polyesters, polysiloxanes, polyamides, polyurethanes, poly(cyclo olefins), epoxies, and random or alternating copolymers thereof; and more specifically, polycarbonates such as poly(4,4′-isopropylidene-diphenylene)carbonate (also referred to as bisphenol-A-polycarbonate), poly(4,4′-cyclohexylidinediphenylene)carbonate (also referred to as bisphenol-Z-polycarbonate), poly(4,4′-isopropylidene-3,3′-dimethyl-diphenyl)carbonate (also referred to as bisphenol-C-polycarbonate), and the like. In embodiments, electrically inactive binders are comprised of polycarbonate resins with a molecular weight of from about 20,000 to about 100,000, or with a molecular weight M_{w} of from about 50,000 to about 100,000 preferred. Generally, the transport layer contains from about 10 to about 75 percent by weight of the hole transport material, and more specifically, from about 35 percent to about 50 percent of this material.

The thickness of each of the charge transport layers in embodiments is from about 5 to about 90 micrometers, but thicknesses outside this range may in embodiments also be selected. The charge transport layer should be an insulator to the extent that an electrostatic charge placed on the hole transport layer is not conducted in the absence of illumination at a rate sufficient to prevent formation and retention of an electrostatic latent image thereon. In general, the ratio of the thickness of the charge transport layer to the photogenerating layer can be from about 2:1 to 200:1, and in some instances 400:1. The charge transport layer is substantially nonabsorbing to visible light or radiation in the region of intended use, but is electrically “active” in that it allows the injection of photogenerated holes from the photoconductive layer, or photogenerating layer, and allows these holes to be transported through itself to selectively discharge a surface charge on the surface of the active layer.

The thickness of the continuous charge transport overcoat layer selected depends upon the abrasiveness of the charging (bias charging roll), cleaning (blade or web), development (brush), transfer (bias transfer roll), and the like in the system employed, and can be up to about 10 micrometers. In embodiments, this thickness for each layer is from about 1 micrometer to about 5 micrometers. Various suitable and conventional methods may be used to mix, and thereafter apply the overcoat layer coating mixture to the photogenerating layer. Typical application techniques include spraying, dip coating, roll coating, wire wound rod coating, and the like. Drying of the deposited coating may be effected by any suitable conventional technique, such as oven drying, infrared radiation drying, air drying, and the like. The dried overcoating layer of this disclosure should transport holes during imaging and should not have too high a free carrier concentration. Free carrier concentration in the overcoat increases the dark decay.

The overcoat layer or layers can comprise the same components as the charge transport layer wherein the weight ratio between the charge transporting small molecule and the suitable electrically inactive resin binder is less, such as for example, from about 0/100 to about 60/40, or from about 20/80 to about 40/60.

Examples of components or materials optionally incorporated into the charge transport layer or at least one charge transport layer to, for example, assist in permitting improved lateral charge, migration (LCM) resistance include hindered phenolic antioxidants, such as tetrakis methylene(3,5-di-tert-butyl-4-hydroxy hydrocinnamate)methane (IRGANOX 1010™, available from Ciba Specialty Chemical), butylated hydroxytoluene (BHT), and other hindered phenolic antioxidants including SUMILIZER BHT-R™, MDP-S™, BBM-S™, WX-R™, NW™, BP-76™, BP-101™, GA-80™, GM™ and GS™ (available from Sumitomo Chemical Co., Ltd.), IRGANOX 1035™, 1076™, 1098™, 1135™, 1141™, 1222™, 1330™, 1425WL™, 1520L™, 245™, 259™, 3114™, 3790™, 5057™ and 565™ (available from Ciba Specialties Chemicals), and ADEKA STAB AO-20™, AO-30™, AO-40™, AO-50™, AO-60™, AO-70™, AO-80™ and AO-330™ (available from Asahi Denka Co., Ltd.); hindered amine antioxidants such as SANOL LS-2626™, LS-765™, LS-770™ and LS-744™ (available from SNKYO CO., Ltd.), TINUVIN 144™ and 622LD™ (available from Ciba Specialties Chemicals), MARK LA57™, LA67™, LA62™, LA68™ and LA63™ (available from Asahi Denka Co., Ltd.), and SUMILIZER TPS™ (available from Sumitomo Chemical Co., Ltd.); thioether antioxidants such as SUMILIZER TP-D™ (available from Sumitomo Chemical Co., Ltd); phosphite antioxidants such as MARK 2112™, PEP-8™, PEP-24G™, PEP-36™, 329K™ and HP-10™ (available from Asahi Denka Co., Ltd.); other molecules such as bis(4-diethylamino-2-methylphenyl)phenylmethane (BDETPM), bis-[2-methyl-4-(N-2-hydroxyethyl-N-ethyl-aminophenyl)]-phenylmethane (DHTPM), and the like. The weight percent of the antioxidant in at least one of the charge transport layers is from about 0 to about 20, from about 1 to about 30, or from about 3 to about 8 weight percent.

A number of processes may be used to mix and thereafter apply the charge transport layer or layers coating mixture to the photogenerating layer. Typical application techniques include spraying, dip coating, roll coating, wire wound rod coating, and the like. Drying of the charge transport deposited coating may be effected by any suitable conventional technique such as oven drying, infrared radiation drying, air drying, and the like.

The thickness of the photoconductor substrate layer depends on many factors, including economical considerations, electrical characteristics, number of layers, components in each of the layers, and the like, thus this layer may be of substantial thickness, for example over about 3,000 microns, and more specifically the thickness of this layer can be from about 1,000 to about 3,000 microns, from about 100 to about 1,000 microns or from about 300 to about 700 microns, or of a minimum thickness. In embodiments, the thickness of this layer is from about 75 microns to about 300 microns, or from about 100 to about 150 microns.

The substrate may be opaque or substantially transparent and may comprise any suitable material having the required mechanical properties. Accordingly, the substrate may comprise a layer of an electrically nonconductive or conductive material such as an inorganic or an organic composition. As electrically nonconducting materials, there may be employed various resins known for this purpose including polyesters, polycarbonates, polyamides, polyurethanes, and the like, which are flexible as thin webs. An electrically conducting substrate may be any suitable metal of, for example, aluminum, nickel, steel, copper, and the like, or a polymeric material, as described above, filled with an electrically conducting substance, such as carbon, metallic powder, and the like, or an organic electrically conducting material. The electrically insulating or conductive substrate may be in the form of an endless flexible belt, a web, a rigid cylinder, a sheet and the like. The thickness of the substrate layer depends on numerous factors, including strength desired and economical considerations. For a drum, as disclosed in a copending application referenced herein, this layer may be of substantial thickness of, for example, up to many centimeters or of a minimum thickness of less than a millimeter. Similarly, a flexible belt may be of substantial thickness of, for example, about 250 micrometers, or of minimum thickness of less than about 50 micrometers, provided there are no adverse effects on the final electrophotographic device.

In embodiments where the substrate layer is not conductive, the surface thereof may be rendered electrically conductive by an electrically conductive coating. The conductive coating may vary in thickness over substantially wide ranges depending upon the optical transparency, degree of flexibility desired, and economic factors.

Illustrative examples of substrates are as illustrated herein, and more specifically layers selected for the imaging members of the present disclosure, and which substrates can be opaque or substantially transparent comprise a layer of insulating material including inorganic or organic polymeric materials, such as MYLAR® a commercially available polymer, MYLAR® containing titanium, a layer of an organic or inorganic material having a semiconductive surface layer, such as indium tin oxide, or aluminum arranged thereon, or a conductive material inclusive of aluminum, chromium, nickel, brass, or the like. The substrate may be flexible, seamless, or rigid, and may have a number of many different configurations, such as for example, a plate, a cylindrical drum, a scroll, an endless flexible belt, and the like. In embodiments, the substrate is in the form of a seamless flexible belt. In some situations, it may be desirable to coat on the back of the substrate, particularly when the substrate is a flexible organic polymeric material, an anticurl layer, such as for example polycarbonate materials commercially available as MAKROLON®, a polycarbonate resin having a weight average molecular weight of from about 50,000 to about 100,000, commercially available from Farbenfabriken Bayer A.G., or similar resin.

The photogenerating layer in embodiments is comprised of, for example, about 60 weight percent of Type V hydroxygallium phthalocyanine or chlorogallium phthalocyanine, and about 40 weight percent of a resin binder like poly (vinyl chloride-co-vinyl acetate) copolymer, such as VMCH (available from Dow-Chemical). Generally, the photogenerating layer can contain known photogenerating pigments, such as metal phthalocyanines, metal free phthalocyanines, alkylhydroxyl gallium phthalocyanines, hydroxygallium phthalocyanines, chlorogallium phthalocyanines, perylenes, especially bis(benzimidazo)perylene, titanyl phthalocyanines, and the like, and more specifically, vanadyl phthalocyanines, Type V hydroxygallium phthalocyanines, and inorganic components such as selenium, selenium alloys, and trigonal selenium. The photogenerating pigment can be dispersed in a resin binder similar to the resin binders selected for the charge transport layer, or alternatively no resin binder need be present. Generally, the thickness of the photogenerating layer depends on a number of factors, including the thicknesses of the other layers and the amount of photogenerating material contained in the photogenerating layer. Accordingly, this layer can be of a thickness of, for example, from about 0.05 micron to about 10 microns, and more specifically, from about 0.25 micron to about 2 microns when, for example, the photogenerating compositions are present in an amount of from about 30 to about 75 percent by volume. The maximum thickness of this layer in embodiments is dependent primarily upon factors, such as photosensitivity, electrical properties and mechanical considerations. The photogenerating layer binder resin is present in various suitable amounts, for example from about 1 to about 50, and more specifically, from about 1 to about 10 weight percent, and which resin may be selected from a number of known polymers, such as poly(vinyl butyral), poly(vinyl carbazole), polyesters, polycarbonates, poly(vinyl chloride), polyacrylates and methacrylates, copolymers of vinyl chloride and vinyl acetate, phenolic resins, polyurethanes, poly(vinyl alcohol), polyacrylonitrile, polystyrene, and the like. It is desirable to select a coating solvent that does not substantially disturb or adversely affect the other previously coated layers of the device. Examples of coating solvents for the photogenerating layer are ketones, alcohols, aromatic hydrocarbons, halogenated aliphatic hydrocarbons, ethers, amines, amides, esters, and the like. Specific solvent examples are cyclohexanone, acetone, methyl ethyl ketone, methanol, ethanol, butanol, amyl alcohol, toluene, xylene, chlorobenzene, carbon tetrachloride, chloroform, methylene chloride, trichloroethylene, tetrahydrofuran, dioxane, diethyl ether, dimethyl formamide, dimethyl acetamide, butyl acetate, ethyl acetate, methoxyethyl acetate, and the like.

The photogenerating layer may comprise amorphous films of selenium and alloys of selenium and arsenic, tellurium, germanium and the like, hydrogenated amorphous silicon and compounds of silicon and germanium, carbon, oxygen, nitrogen and the like fabricated by vacuum evaporation or deposition. The photogenerating layers may also comprise inorganic pigments of crystalline selenium and its alloys; Group II to VI compounds; and organic pigments such as quinacridones, polycyclic pigments such as dibromo anthanthrone pigments, perylene and perinone diamines, polynuclear aromatic quinones, azo pigments including bis-, tris- and tetrakis-azos; and the like dispersed in a film forming polymeric binder and fabricated by solvent coating techniques: and a number of phthalocyanines, like a titanyl phthalocyanine, titanyl phthalocyanine Type V; oxyvanadium phthalocyanine, chloroaluminum phthalocyanine, copper phthalocyanine, oxytitanium phthalocyanine, chlorogallium phthalocyanine, hydroxygallium phthalocyanine magnesium phthalocyanine and metal free phthalocyanine and the like with infrared sensitivity photoreceptors exposed to low-cost semiconductor laser diode light exposure devices.

In embodiments, examples of polymeric binder materials that can be selected as the matrix for the photogenerating layer are illustrated in U.S. Pat. No. 3,121,006, the disclosure of which is totally incorporated herein by reference. Examples of binders are thermoplastic and thermosetting resins, such as polycarbonates, polyesters, polyamides, polyurethanes, polystyrenes, polyarylethers, polyarylsulfones, polybutadienes, polysulfones, polyethersulfones, polyethylenes, polypropylenes, polyimides, polymethylpentenes, poly(phenylene sulfides), poly(vinyl acetate), polysiloxanes, polyacrylates, polyvinyl acetals, polyamides, polyimides, amino resins, phenylene oxide resins, terephthalic acid resins, phenoxy resins, epoxy resins, phenolic resins, polystyrene and acrylonitrile copolymers, poly(vinyl chloride), vinyl chloride and vinyl acetate copolymers, acrylate copolymers, alkyd resins, cellulosic film formers, poly(amideimide), styrenebutadiene copolymers, vinylidene chloride-vinyl chloride copolymers, vinyl acetate-vinylidene chloride copolymers, styrene-alkyd resins, poly(vinyl carbazole), and the like. These polymers may be block, random or alternating copolymers.

The coating of the photogenerating layer in embodiments of the present disclosure can be accomplished with spray, dip or wire-bar methods such that the final dry thickness of the photogenerating layer is as illustrated herein, and can be, for example, from about 0.01 to about 30 microns after being dried at, for example, about 40° C. to about 150° C. for about 15 to about 90 minutes. More specifically, photogenerating layer of a thickness, for example, of from about 0.1 to about 30, or from about 0.5 to about 2 microns can be applied to or deposited on the substrate, on other surfaces in between the substrate and the charge transport layer, and the like. The photogenerating composition or pigment is present in the resinous binder composition in various amounts. From about 5 percent by volume to about 90 percent by volume of the photogenerating pigment is dispersed in about 10 percent by volume to about 95 percent by volume of the resinous binder, or from about 20 percent by volume to about 30 percent by volume of the photogenerating pigment is dispersed in about 70 percent by volume to about 80 percent by volume of the resinous binder composition. In one embodiment, about 10 percent by volume of the photogenerating pigment is dispersed in about 90 percent by volume of the resinous binder composition.

Various suitable and conventional known processes may be used to mix, and thereafter apply the photogenerating layer coating mixture, like spraying, dip coating, roil coating, wire wound rod coating, vacuum sublimation, and the like. For some applications, the photogenerating layer may be fabricated in a dot or line pattern. Removal of the solvent of a solvent-coated layer may be effected by any known conventional techniques such as oven drying, infrared radiation drying, air-drying and the like.

A charge blocking layer or hole blocking layer may optionally be applied to the electrically conductive surface prior to the application of a photogenerating layer. When desired, an adhesive layer may be included between the charge blocking layer, the hole blocking layer or interfacial layer and the photogenerating layer. Usually, the photogenerating layer is applied onto the blocking layer and a charge transport layer or plurality of charge transport layers are formed on the photogenerating layer. This structure may have the photogenerating layer on top of or below the charge transport layer.

The hole blocking layer can be, for example, comprised of from about 20 weight percent to about 80 weight percent, and more specifically, from about 55 weight percent to about 65 weight percent of a suitable component like a metal oxide, such as TiO₂, from about 20 weight percent to about 70 weight percent, and more specifically, from about 25 weight percent to about 50 weight percent of a phenolic resin; from about 2 weight percent to about 20 weight percent and, more specifically, from about 5 weight percent so about 15 weight percent of a phenolic compound containing at least two phenolic groups, such as bisphenol S, and from about 2 weight percent to about 15 weight percent, and more specifically, from about 4 weight percent to about 10 weight percent of a plywood suppression dopant, such as SiO₂. The hole blocking layer coating dispersion can, for example, be prepared as follows. The metal oxide/phenolic resin dispersion is first prepared by ball milling or dynomilling until the median particle size of the metal oxide in the dispersion is less than about 10 nanometers, for example from about 5 to about 9. To the above dispersion are added a phenolic compound and dopant followed by mixing. The hole blocking layer coating dispersion can be applied by dip coating or web coating, and the layer can be thermally cured after coating. The hole blocking layer resulting is, for example, of a thickness of from about 0.01 micron to about 30 microns, and more specifically, from about 0.1 micron to about 8 microns. Examples of phenolic resins include formaldehyde polymers with phenol, p-tert-butylphenol, cresol, such as VARCUM™ 29159 and 29101 (available from OxyChem Company), and DURITE™ 97 (available from Borden Chemical); formaldehyde polymers with ammonia, cresol and phenol, such as VARCUM™ 29112 (available from OxyChem Company); formaldehyde polymers with 4,4′-(1-methylethylidene)bisphenol, such as VARCUM™ 29108 and 29116 (available from OxyChem Company); formaldehyde polymers with cresol and phenol, such as VARCUM™ 29457 (available from OxyChem Company), DURITE™ SD-423A, SD-422A (available from Borden Chemical); or formaldehyde polymers with phenol and p-tert-butylphenol, such as DURITE™ ESD 556C (available from Border Chemical).

The optional hole blocking layer may be applied to the substrate. Any suitable and conventional blocking layer capable of forming an electronic barrier to holes between the adjacent photoconductive layer (or electrophotographic imaging layer) and the underlying conductive surface of substrate may be selected.

The optional hole blocking or undercoat layers for the imaging members of the present disclosure can contain a number of components including known hole blocking components, such as amino silanes, doped metal oxides, TiSi, a metal oxide like titanium, chromium, zinc, tin and the like; a mixture of phenolic compounds and a phenolic resin or a mixture of two phenolic resins, and optionally a dopant such as SiO₂. The phenolic compounds usually contain at least two phenol groups, such as bisphenol A (4,4′-isopropylidenediphenol), E (4,4′-ethylidenebisphenol), F (bis(4-hydroxyphenyl)methane), M (4,4′-(1,3-phenylenediisopropylidene)bisphenol), P (4,4′-(1,4-phenylene diisopropylidene)bisphenol), S (4,4′-sulfonyldiphenol), and Z (4,4′-cyclohexylidenebisphenol); hexafluorobisphenol A (4,4′-(hexafluoro isopropylidene)diphenol), resorcinol, hydroxyquinone, catechin, and the like.

In embodiments, a suitable known adhesive layer can be included in the photoconductor. Typical adhesive layer materials include, for example, polyesters, polyurethanes, and the like. The adhesive layer thickness can vary and in embodiments is, for example, from about 0.05 micrometer (500 Angstroms) to about 0.3 micrometer (3,000 Angstroms). The adhesive layer can be deposited on the hole blocking layer by spraying, dip coating, roll coating, wire wound rod coating, gravure coating, Bird applicator coating, and the like. Drying of the deposited coating may be effected by, for example, oven drying, infrared radiation drying, air drying and the like.

As optional adhesive layers usually in contact with or situated between the hole blocking layer and the photogenerating layer, there can be selected various known substances inclusive of copolyesters, polyamides, poly(vinyl butyral), poly(vinyl alcohol), polyurethane and polyacrylonitrile. This layer is, for example, of a thickness of from about 0.001 micron to about 1 micron, or from about 0.1 to about 0.5 micron. Optionally, this layer may contain effective suitable amounts, for example from about 1 to about 10 weight percent, of conductive and nonconductive particles, such as zinc oxide, titanium dioxide, silicon nitride, carbon black, and the like, to provide, for example, in embodiments of the present disclosure further desirable electrical and optical properties.

Primarily for purposes of brevity, the examples of each of the substituents and each of the components/compounds/molecules, polymers, (components) for each of the layers, specifically disclosed herein are not intended to be exhaustive. Thus, a number of components, polymers, formulas, structures, and R group or substituent examples and carbon chain lengths not specifically disclosed or claimed are intended to be encompassed by the present disclosure and claims. For example, these substituents include suitable known groups, such as aliphatic and aromatic hydrocarbons with various carbon chain lengths, and which hydrocarbons can be substituted with a number of suitable known groups and mixtures thereof. Also, the carbon chain lengths are intended to include all numbers between those disclosed or claimed or envisioned, thus from 1 to about 20 carbon atoms, includes 1, 2, 3, 4, 5, 6, 7, 8, 9, 10, 11, 12, 13, 14, 15 up to 20 or more. Similarly, the thickness of each of the layers, the examples of components in each of the layers, the amount ranges of each of the components disclosed and claimed is not exhaustive, and it is intended that the present disclosure and claims encompass other suitable parameters not disclosed or that may be envisioned.

At least one, especially as applicable to the charge transport layer, refers for example, to 1 layer, 2 to about 7 layers, 1 to about 5 layers, 1 to 2 layers and the like.

The following Examples are being submitted to illustrate embodiments of the present disclosure. These Examples are intended to be illustrative only, and are not intended to limit the scope of the present disclosure. Also, parts and percentages are by weight unless otherwise indicated. Comparative Examples and data are also provided.

### EXAMPLES

### N,N,N′N′-tetra(4-methylphenyl)-(1,1′-biphenyl)-4,4′-diamine (Compound 1)

The purification procedures to produce N,N,N′N′-tetra(4-methylphenyl)-(1,1′-biphenyl)-4,4′-diamine with a purity of 98 to 100 percent could include train sublimation, a Kaufmann column run with alumina and a non-polar solvent such as hexane, hexanes, cyclohexane, heptane and the like, absorbent treatments such as with the use of alumina, clay, charcoal and the like and recrystallization to produce the desired purity.

The compound can be prepared through other reactions such as a Buchwald-Hartwig reaction and any other obvious reactions to those skilled in the art which would produce the desired compound. The purity of the final material may be instrumental in obtaining the improved electrical and mechanical properties.

### Example 1

An imaging or photoconducting member incorporating Compound 1was prepared in accordance with the following procedure. A metallized mylar substrate was provided and a HOGaPc/poly(bisphenol-Z carbonate) photogenerating layer was machine coated over the substrate. The photogenerating layer was overcoated with a charge transport layer prepared by introducing into an amber glass bottle 35 weight percent of high quality N,N,N′N′-tetra(4-methylphenyl)-(1,1′-biphenyl)-4,4′-diamine (Compound 1), synthesized as discussed above, having a purity of from about 99 to about 100 percent as determined by HPLC and NMR and 65 weight percent of MAKROLON 5705®, a known polycarbonate resin having a molecular weight average of from about 50,000 to about 100,000, commercially available from Farbenfabriken Bayer A.G. The resulting mixture was then dissolved in methylene chloride to form a solution containing 15 percent by weight solids. This solution was applied on the photogenerating layer to form a layer coating that upon drying (120° C. for 1 minute) had a thickness of 30 microns. During this coating process, the humidity was equal to or less than about 15 percent.

A photoconductor is prepared by repeating the process of Example 1 except that the charge transport layer is prepared by introducing into an amber glass bottle 50 weight percent of high quality N,N,N′N′-tetra(4-methylphenyl)-(1,1′-biphenyl)-4,4′-diamine (Compound 1) and about 50 weight percent MAKROLON 5705.

### Comparative Example 1

A comparative photoconductor is prepared by repeating the process of Example 1 except that the charge transport layer is prepared by introducing into an amber glass bottle 50 weight percent of N,N′-diphenyl-N,N-bis(3-methylphenyl)-1,1′-biphenyl-4,4′-diamine (Compound 2) of a purity of from about 99 percent to about 100 percent, and about 50 weight percent MAKROLON 5705.

### Comparative Example 2

A comparative photoconductor is prepared by repeating the process of Example 1 except that the charge transport layer is prepared by introducing into art amber glass bottle 50 weight percent of standard or low quality N,N,N′N′-tetra(4-methylphenyl)-(1,1′-biphenyl)-4,4′-diamine (Compound 3) and about 50 weight percent MAKROLON 5705.

Electrical Property Testing:

The xerographic electrical properties of the above prepared photoconductors were determined by known means, such as by charging the surfaces thereof with a corona discharge source until the surface potentials, as measured by a capacitively coupled probe attached to an electrometer, attained an initial value V₀ of about −800 volts. After resting for a 0.5 second in the dark, the charged members attained a surface potential of V_{ddp}, dark development potential. The photoconductive imaging members were then exposed to light from a filtered Xenon lamp with a 150 watt bulb, thereby inducing a photodischarge which resulted in a reduction of surface potential to a V_{bg} value, background potential. The wavelength of the incident light was 780 nanometers, and the exposure energy of the incident light varied from 0 to 10 ergs/cm². By plotting the surface potential against exposure energy, a photodischarge curve was constructed. The photosensitivity of the imaging member can be described in terms of E_{1/2}, (half-discharge exposure energy), that is the amount of exposure energy in erg/cm² required to achieve 50 percent photodischarge from the dark development potential. Residual potential after erase Vr was measured after the device was further subjected to a high intensity white light irradiation from a secondary filtered xenon lamp. The cyclic stability of the devices was assessed by performing repetitive charging and discharging over 10,000 cycles. The expose energy was kept at a constant value of 20 ergs/cm² and the cycle time was 1 cycle per second. The voltage after each charge expose cycle is measured and recorded as the V_{low}. The changes in V_{low} were monitored by subtracting the initial voltages at 100cycle from the final voltages of last cycle. The smaller the changes the better is the cyclic stability, another important attribute for a functional devices. The results obtained for the photoconductive members fabricated in accordance with the above examples are summarized in table 1.

As illustrated in Table 1, both imaging members from Example 1 containing high quality N,N,N′N′-tetra(4-methylphenyl)-(1,1′-biphenyl)-4,4′-diamine (Compound 1) exhibited improved electrical properties when compared to imaging members containing N,N′-diphenyl-N,N-bis(3-methylphenyl)-1,1′-biphenyl-4,4′-diamine (Compound 2) and standard quality N,N,N′N′-tetra(4-methylphenyl)-(1,1′-biphenyl)-4,4′-diamine (Compound 3)

TABLE 1

| | | E½ | Vᵣ | |
|---|---|---|---|---|
| Sample | Weight % | ergs/cm² | volts | V_{low100}–V_{low10k} |
| Compound 1 | 50 | 1.23 | 2 | −1 |
| Compound 1 | 50 | 1.25 | 4 | 0 |
| Compound 2 | 50 | 1.22 | 22 | 14 |
| Compound 3 | 50 | 1.23 | 18 | 6 |

Deletion Resistance:

Deletion resistance was evaluated by a lateral charge migration (LCM) print testing scheme. The above prepared hand coated photoconductor devices were cut into 6″×1″ strips. One end of the strip from the respective devices was cleaned using a solvent to expose the metallic conductive layer on the substrate. The conductivity of the exposed metallic TiZr conductive layer was then measured to ensure that the metal had not been removed during cleaning. The conductivity of the exposed metallic TiZr conductive layer was measured using a multimeter to measure the resistance across the exposed metal layer (around 1 KOhm). A fully operational 85 mm DC12 A Xerox Corporation standard Docu Color photoreceptor drum was prepared to expose a lengthwise strip of bare aluminum (0.5″×12″) to provide the ground for the handcoated device when it is operated. The cleaning blade was removed from the drum housing to prevent it from removing the handcoated devices during operation.

The hand coated imaging member from Example I with N,N,N′N′-tetra(4-methylphenyl)-(1,1′-biphenyl)-4,4′-diamine at 35 weight percent and the comparative control device 1 were then mounted onto a photoreceptor drum using conductive copper tape to adhere the exposed conductive end of the devices to the exposed aluminum strip on the drum to complete a conductive path to the ground. After mounting the devices, the device-to-drum conductivity was measured using a standard multimeter in a resistance mode. The resistance between the respective devices and the drum should be similar to the resistance of the conductive coating on the respective hand coated devices. The ends of the devices are then secured to the drum using scotch tape, and all exposed conductive surfaces were covered with scotch tape. The drum was then placed in a Docu-color 12 (DC12) machine and a template containing 1 bit, 2 bit, 3 bit, 4 bit, and 5 bit lines was printed. The machine settings (developer bias, laser power, grid bias.) were adjusted to obtain visible print that resolved the 5 individual lines above. If the 1 bit line is barely showing, then the settings are saved and the print becomes the reference, or the pre-exposure print. The drum was removed and placed in charge-discharge apparatus generates corona discharge during operation. The drum was charged and discharged (cycled) for 20,000 cycles to induce deletion (LCM). The drum was then removed from the apparatus and placed in the DC12 machine and the template was printed again.

The imaging member from Example I with N,N,N′N′-tetra(4-methylphenyl)-(1,1′-biphenyl)-4,4′-diamine at 35 weight percent in the charge transport layer shows a high level of deletion resistance after 20,000 cycles. The 1 bit, 2 bit, 3 bit, 4 bit, 5 bit lines are all visible when printed. The comparative sample show a much lower level of deletion resistance after 20,000 cycles.

Discharge Rate:

Discharge rate was evaluated by measuring the surface potential of the photoconductor at specified time intervals after photodischarge. Discharge rate was determined by electrostatically charging the surfaces of the imaging members with a corona discharging device, in the dark, until the surface potential attained an initial value of about 500 volts, as measured by a ESV probe attached to an electrometer. The devices are tested with the exposure light having a measured energy of 2 ergs/cm² and a wavelength of 780 nm, from a filtered xenon lamp. A reduction in the surface potential due to photodischarge effect was measured at 33, 117, 234, 468, and 702 milliseconds after photodischarge.

TABLE 2

| | Photoconductor Surface Potential Measured Voltage @ 2 ergs/cm² | |
|---|---|---|
| | | Compound 3 [Comparative Example 2]. |
| | | Photoconductor containing N,N,N′N′-tetra(4- |
| | Compound 1. Photoconductor containing | methylphenyl)-(1,1′-biphenyl)-4,4′-diamine |
| | high quality N,N,N′N′-tetra(4-methylphenyl)- | of a quality obtained by previous methods in |
| | (1,1′-biphenyl)-4,4′-diamine in the CTL, the | the CTL, the CTL having 50 weight % |
| Time After Exposure | CTL having 50 weight % Compound 1 to 50 | Compound 1 to 50 weight % |
| (Milliseconds) | weight % MAKROLON 5705 ® | MAKROLON 5705 ® |
| 702 | 23 | 52 |
| 468 | 24 | 63 |
| 234 | 33 | 107 |
| 117 | 40 | 167 |
| 33 | 57 | 205 |

As illustrated in Table 2, the photoconductor containing Compound 1, having high quality, exhibited superior discharge properties. For example, at 33, 234, and 702 milliseconds after photodischarge, respectively, the photoconductor containing Compound 1 exhibited surface potential measured voltages of 57 v, 33 v, and 23 v at 2 ergs/cm²; whereas the photoreceptor of Comparative Example 2 exhibited surface potential measured voltages of 205 v, 107 v, and 52 v at 2 ergs/cm², respectively. Thus, the high quality electrical characteristics of Compound 1 are highly desirable.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A hole transport material comprising a substituted biphenyl diamine of the following general formula 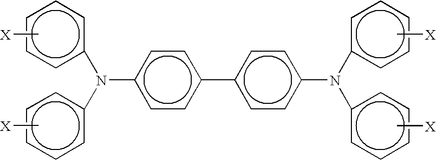
wherein each X is independently selected from the group consisting of —H, alkyl(—CₙH₂ₙ₊₁) where n is from 1 to about 10, aralkyl, and aryl groups, the aralkyl and aryl groups having, for example, from about 5 to about 30 carbon atoms; and wherein
when incorporated into a photoreceptor, the photoreceptor will discharge from about 85% to about 100% of its surface potential in from 0 to about 40 milliseconds upon being subjected to xerographic charging and exposure to radiant energy of from about 1 erg/cm² to about 5 ergs/cm².

2. The hole transport material of claim 1, wherein when incorporated into a photoreceptor, the photoreceptor will discharge from about 85% to about 100% of its surface potential in from about 0 to about 40 milliseconds of being subjected to xerographic charging and exposure to radiant energy of about 2 ergs/cm².

3. The hole transport material of claim 1, wherein when incorporated into a photoreceptor, the photoreceptor will discharge from about 90% to about 100% of its surface potential within from about 0 to about 40 milliseconds of being subjected to xerographic charging and exposure to radiant energy of about 2 ergs/cm².

4. The hole transport material of claim 1, wherein the xerographic charging voltage is from about 400 volts to about 600 volts and the radiant energy is from about 1 erg/cm² to about 4 ergs/cm².

5. The hole transport material of claim 1, wherein the xerographic charging voltage is about 450 volts to about 550 volts and the radiant energy is from about 1 erg/cm² to about 3 ergs/cm².

6. The hole transport material of claim 1, wherein each X independently represents alkyl of from 1 to about 15 carbon atoms.

7. The hole transport material of claim 1, having a purity of from about 95 percent to about 100 percent.

8. The hole transport material of claim 1, having a purity of from about 98 percent to about 100 percent.

9. The hole transport material of claim 1, wherein the substituted biphenyl diamine is one of N,N,N′N′-tetra(4-methylphenyl)-(1,1′-biphenyl)-4,4′-diamine; N,N,N′N′-tetra(4-ethylphenyl)-(1,1′-biphenyl)-4,4′-diamine; N,N,N′N′-tetra(4-propylphenyl)-(1,1′-biphenyl)-4,4′-diamine; and N,N,N′N′-tetra(4-butylphenyl)-(1,1′-biphenyl)-4,4′-diamine.

10. A photoreceptor comprising a supporting substrate, a photogenerating layer thereover and a charge transport layer comprising a hole transport material comprising at least one substituted biphenyl diamine of the following general formula 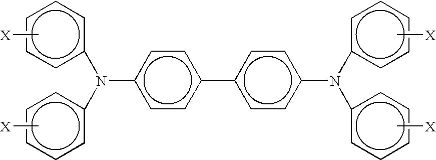
wherein each X is independently selected from the group consisting of —H, alkyl(—CₙH₂ₙ₊₁) where n is from 1 to about 10, aralkyl, and aryl groups, the aralkyl and aryl groups having from about 5 to about 30 carbon atoms; and wherein
when incorporated into a photoreceptor, the photoreceptor will discharge from about 85% to about 100% of its surface potential in from 0 to about 40 milliseconds upon being subjected to xerographic charging and exposure to radiant energy of from about 1 erg/cm² to about 5 ergs/cm².

11. A photoreceptor according to claim 10, wherein the photoreceptor will discharge from about 85% to about 100% of its surface potential in from about 0 to about 40 milliseconds of being subjected to xerographic charging and exposure to radiant energy of about 2 ergs/cm².

12. A photoreceptor according to claim 10, the photoreceptor will discharge from about 90% to about 100% of its surface potential within from about 0 to about 40 milliseconds of being subjected to xerographic charging and exposure to radiant energy of about 2 ergs/cm².

13. A photoreceptor according to claim 10, wherein the xerographic charging voltage is from about 400 volts to about 600 volts and the radiant energy is from about 1 erg/cm² to about 4 ergs/cm².

14. A photoreceptor according to claim 10, wherein the xerographic charging voltage is about 450 volts to about 550 volts and the radiant energy is from 1 ergs/cm² to about 3 ergs/cm².

15. A photoreceptor according to claim 10, wherein each X independently represents alkyl of from 1 to about 15 carbon atoms.

16. A photoreceptor according to claim 10, wherein the hole transport material is present in an amount of from about 1 to about 75 percent by weight.

17. A photoreceptor according to claim 10, wherein the hole transport material is present in an amount of from about 25 to about 75 percent by weight.

18. A photoreceptor according to claim 10, wherein the purity of the hole transport material is from about 95 percent to about 100 percent and the hole transport material is present in an amount of from about 30 to about 65 percent by weight.

19. A photoreceptor according to claim 10, wherein the purity of the hole transport material is from about 98 percent to about 100 percent and the hole transport material is present in an amount of from about 40 to about 60 percent by weight.

20. A photoreceptor according to claim 10, wherein the hole transport material is one of N,N,N′N′-tetra(4-methylphenyl)-(1,1′-biphenyl)-4,4′-diamine; N,N,N′N′-tetra(4-ethylphenyl)-(1,1′-biphenyl)-4,4′-diamine; N,N,N′N′-tetra(4-propylphenyl)-(1,1′-biphenyl)-4,4′-diamine: and N,N,N′N′-tetra(4-butylphenyl)-(1,1′-biphenyl)-4,4′-diamine.

21. A photoreceptor according to claim 10, wherein said alkyl is methyl, ethyl, propyl, butyl, pentyl, hexyl, isomers thereof or mixtures thereof and wherein the purity of said component is from about 98 percent to about 100 percent and said component is present in an amount of from about 10 to about 60 percent by weight.

22. A photoreceptor according to claim 10, wherein said member further includes at least one of a hole blocking layer and an adhesive layer.

23. A photoreceptor according to claim 10, wherein said photoconductor is a flexible belt.
